(19) United States
(12) Patent Application Publication
(10) Pub. No.: US 2003/0177380 A1
(43) Pub. Date: Sep. 18, 2003

(54) Devices for storing array data and methods of using the same

(76) Inventor: Stanley P. Woods, Cupertino, CA (US)

Correspondence Address: Agilent Technologies, Inc., Legal Department, DL429, Intellectual Property Administration, P.O. Box 7599, Loveland, CO, 80537-0599, US

(21) Appl. No.: 10/101,458

(22) Filed: Mar. 18, 2002

(51) Int. Cl. G05B 11/01; G06F 19/00; G01N 33/48; G01N 33/50

(52) U.S. Cl. 713/200; 702/20

(57) ABSTRACT

Devices and methods for storing data related to at least one array are provided. The subject devices are characterized by an array holder that holds a substrate having at least one array and a data storage element operatively coupled thereto and configured to receive and store data related to a held array. Stored data may include calibration data and/or the number of times the array is scanned. The data storage element may be configured to communicate with at least one external apparatus using a physical connection. In using the subject devices, a subject array holder holds a substrate having at least one array and data related to the at least one array is received and stored by the data storage element. The data storage element may communicate with at least one external apparatus using a physical connection. Also provided are kits for practicing the subject methods.

### FIELD OF THE INVENTION

The field of this invention is arrays, specifically biopolymeric arrays and more specifically devices to receive and store information related to biopolymeric arrays.

### BACKGROUND OF THE INVENTION

“Biochips” or arrays (also known as microarrays) of binding agents, such as oligonucleotides, cDNA and peptides, and the like have become an increasingly important tool in the biotechnology industry and related fields. These binding agent arrays, in which a plurality of binding agents are deposited onto a solid support surface in the form of an “array” or pattern, find use in a variety of applications, including gene expression analysis, drug screening, nucleic acid sequencing, mutation analysis, and the like.

In general, an array is subjected to at least three distinct processes after it has been manufactured. The first is the transport of the array from the site of manufacture to the user. The second is the array assay such as an array hybridization assay where the array is reacted with a sample to promote binding of one or more analytes in the sample to the array. The third process includes reading or scanning the array to interrogate it regarding certain information such as specific analyte binding information. Each of these processes involves specific process parameters or conditions to which the array is exposed, such as specific reagents and corresponding volumes used, duration of processes, temperatures, humidity, the number of times the array is scanned, calibration information of ancillary devices, etc.

In array-based assays such as an array hybridization assay, an array of binding agents is reacted with one or more analytes such as polynucleotide analytes, receptor proteins or antiligant molecules, etc., under conditions that promote specific binding of the analyte molecules to one or more of the bound array members or probes. Typically, the goal is to identify one or more position-addressable members of the library array which bind to the analyte as a method of screening for array compounds which bind the analyte. Usually, the analyte is labeled with a detectable reporter such as a fluorescent agent which, in effect, can fluorescently label the one or more array regions where array analyte binding has occurred.

Once the binding of the analyte to one or more array members has occurred, the array is scanned or “read”, usually by radiometric or optical means. A variety of scanning devices have been proposed for scanning such arrays (see for example U.S. Pat. Nos. 5,324,633 and 5,585,639, the disclosures of which are herein incorporated by reference). The array scanning devices typically include a light source, e.g., a laser, photodiode or the like, for transmitting light onto the array and a detector, e.g., a photomultiplier or the like, for detecting a parameter of the transmitted light, e.g., light absorption, fluorescence, etc.

It would be advantageous to be able to easily receive and store information about these arrays, particularly the processes to which the arrays are subjected, to assist in the evaluation thereof, such as for quality control purposes, to set downstream process parameters and to interpret scanning or interrogation results. For example, it would be advantageous to be able to store “static” information relating to the specific array configuration or layout information, such as the array's serial number, expiration date, date of manufacture, and the like. Indeed, it would be most advantageous not only to store static information about the array, but also to automatically collect, receive and store “dynamic” or post-fabrication information regarding the processing of the array, etc.

Currently, bar codes are used to store limited information about an array. For example, such bar codes are oftentimes positioned on the array itself, more specifically on the substrate adjacent an array, and include static information such as serial numbers and information relating to array layout information (see for example U.S. Pat. No. 6,180,351). However, while effective at storing certain data, bar codes have some limitations.

First and foremost, bar codes are not capable of collecting dynamic information. In fact, if information is to be added after the bar code has been configured, such as information related to the processing of the array, or if information must be changed or erased, the bar code must be replaced by another bar code which has had the new information transposed thereupon. In other words, information contained on bar codes is fixed as of the time the bar codes are made and placed on the array, which is typically at the point of fabrication. Second, the bar codes are limited to the amount of information they can store because of size constraints. For example, unique identifiers which match an array to its specific layout information, often referred to as “Globally Unique Identifiers” or GUIDs” or “Universally Unique Identifiers” or “UUIDs”(see for example U.S. Pat. Nos. 5,812,793 and 5,404,523) typically require 128 bit data string. However, a string of such a length when written as a bar code would usually take up about 3 to 4 cm, which is more room than is often available on a substrate adjacent a typical array (which may be less than about 1 cm in any dimension). Thus, oftentimes a second, shorter code is used, where such a shorter code is used to identify the actual unique identifier. However, this technique adds complexity to the array process. Third, a bar code requires the use of a bar code scanner for reading the information contained on the bar code itself. Such ancillary equipment adds to the cost and complexity of data retrieval.

As such, there is continued interest in the development of new devices for receiving and storing data relating to arrays and methods of using the same. Of particular interest would be the development of such a device, and methods of use thereof, that can collect, receive and store both static and dynamic data, particularly calibration related data and the number of times the array has been scanned, can do so automatically, store a large amount of such data, store data in a secure format, reliably, securely and automatically communicate with at least one external apparatus and/or sites to receive data from or transfer data to the external apparatus and activate and/or deactivate at least one external apparatus or component thereof automatically. Relevant Literature

U.S. Patent documents of interest include U.S. Pat. Nos. 5,324,633; 5,404,523; 5,585,639; 5,812,793; 5,837,832; 5,874,219; 5,961,923; 6,019,449; 6,114,122; 6,199,292, 6,125,420; 6,180,351, 6,238,910 and 6,258,593. Also of interest is GB 2319838.

### SUMMARY OF THE INVENTION

Devices and methods for storing data related to at least one array are provided. The subject devices are characterized by an array holder that holds a substrate having at least one array and a data storage element operatively coupled thereto and configured to receive and store data related to a held array. Stored data may include, but is not limited to, calibration data and/or the number of times the array is scanned. The data storage element may be configured to communicate with at least one external apparatus using a physical connection. In using the subject devices, a subject array holder holds a substrate having at least one array and data related to the at least one array is received and stored by the data storage element.

The data storage element may communicate with at least one external apparatus using a physical connection. Also provided are kits for practicing the subject methods.

### BRIEF DESCRIPTIONS OF THE DRAWINGS

FIG. 1A shows an exemplary embodiment of a subject array holder operatively coupled to an external apparatus.

FIG. 1B shows an exemplary embodiment of a subject array holder operatively coupled to an external apparatus.

FIG. 2 shows an exemplary embodiment of a substrate having at least one array associated therewith where the substrate includes a data storage element.

FIG. 3 shows an exemplary substrate having at least one array associated therewith, such as may be used in the devices of the subject invention.

FIG. 4 shows an enlarged view of a portion of FIG. 3 showing spots or features.

FIG. 5 is an enlarged view of a portion of the substrate of FIG. 4.

### DEFINITIONS

The terms “array” “biopolymeric array” and “biomolecular array” are used herein interchangeably to refer to an arrangement of ligands or molecules of interest on a substrate surface which can be used for analyte detection, combinatorial chemistry, or other applications wherein a two-dimensional arrangement of molecules of interest can be used. That is, the terms refer to an ordered pattern of probe molecules adherent to a substrate, i.e., wherein a plurality of molecular probes are bound to a substrate surface and arranged in a spatially defined and physically addressable manner. Such arrays may be comprised of oligonucleotides, peptides, polypeptides, proteins, antibodies, or other molecules used to detect sample molecules in a sample fluid.

The term “array holder” as used herein refers to any device configured to hold a substrate having at least one array.

The term “binding agent” as used herein refers to any agent that is a member of a specific binding pair, where such agents include: polypeptides, e.g. proteins or fragments thereof; nucleic acids, e.g., oligonucleotides, polynucleotides, and the like, as well as other biomolecules, e.g., polysaccharides, etc. The binding of such binding pairs may be permanent or temporary.

The term “data storage element” as used herein refers to any suitable device which is capable of receiving and storing large amounts of data including, but not limited to, magnetic, silicon chip, optical or solid state storage devices (including magnetic or optical disks or tape or RAM, or any other suitable device). In other words, the data storage element is capable of storing a greater amount of data than would be feasible to store on an array bar code and will typically have a storage capacity of from one byte to hundreds of bytes of data to multiple tens or even hundreds of megabytes of data or more. As such, typically about 100 bytes to about 500 megabytes of data or more may be stored, usually from about 250 bytes to about 15 megabytes of data may be stored and more usually from about 0.125 megabytes to about 4 megabytes of data may be stored by the data storage element. Data may be stored in the data storage element manually, for example in the case of static data, or automatically, for example in the case of dynamic data. The stored data may be organized into separate or discrete areas. For example, data may be stored in areas that are generally or broadly accessible and/or stored in areas that are secure or protected, i.e., areas that have limited accessibility, e.g., the areas are protected and accessible only if a password is provided, or the like. Furthermore, the data may be stored in a variety of formats, including, but not limited to, raw, processed, encrypted and decrypted formats. In certain embodiments of the subject invention, certain data may be stored in a generally accessible area and certain other data may be stored in a limited access area, where some or all of the data stored in either or both of the generally accessible areas and/or limited access areas may be raw and/or processed and/or encrypted and/or decrypted.

The term “dynamic data” refers to data that is received and stored during the transport and processing of the array, where such data is received and stored substantially concurrently with the occurrence of the process or stored in “real time” in regards to the transport and process. In other words, dynamic data is data relating to array events, conditions, occurrences or processes to which the array is subject, where such data is stored or recorded at substantially the same time as the array events, conditions, occurrences or processes happen.

Examples of dynamic data include, but are not limited to, conditions or parameters of array transport or delivery or distribution (e.g., temperature, humidity, date and/or time and/or duration of transport, etc., during the transport or delivery or distribution), conditions or parameters relating to an array assay such as an array hybridization assay and/or array scanning processes, (e.g., temperature data, humidity data, identification information relating to external devices such as lot number, part number, manufacturer, etc., gas concentration, duration of processes or steps, reagent data (e.g., manufacturer, stringency, rate of dispensation, amounts, etc.), identification of operator, the number of times the array is scanned, etc.), specific tag or label information, calibration information of ancillary apparatuses, etc.

Although typically dynamic data is data related to post-manufacturing, in certain embodiments dynamic data may include data related to the conditions or parameters of the manufacturing process of the array as well, for example manufacturing conditions such as waveforms, waveform firing parameters, specific fluids used in the manufacturing process (e.g., manufacturer, concentration, lot number, back pressures in the manufacturing vacuum system, etc.), where such data is received and stored substantially concurrently with the manufacturing process. Dynamic data may also include data related to quality control processes, where such data may be used to determine if specific array elements are faulty.

Dynamic data may be received and stored in a data storage element manually such as with a keyboard or computer mouse, etc., or automatically, where the data will typically be received and stored automatically.

The term “polymer” as used herein refers to any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

The term “peptide” as used herein refers to any compound produced by amide formation between a carboxyl group of one amino acid and an amino group of another group, where the amino acids may be naturally occurring or synthetic.

The term “oligopeptide” as used herein refers to peptides with fewer than about 10 to 20 residues, i.e. amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick rigid bottom surface pairing interactions.

The terms “ribonucleic acid” and “RNA” as used herein refers to a polymer composed of ribonucleotides.

The terms “deoxyribonucleic acid” and “DNA” as used herein refers to a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

The terms “measuring sensor” and “sensor” used herein interchangeably herein refer to any device capable of measuring and/or monitoring a condition or parameter such as an environmental condition or parameter and includes, but is not limited to, devices for measuring and/or monitoring temperature such as temperature sensors, e.g., a thermistors and the like, devices for measuring and/or monitoring humidity such as humidity sensors or the like, devices for measuring and/or monitoring volumes such as volume sensors or the like, devices for measuring and/or monitoring the number of times an array has been scanned such as optical sensors or the like, devices for measuring and/or monitoring calibration data, devices for measuring and/or monitoring fluidic rates, devices for measuring and/or monitoring process duration, etc.

The term “static data” as used herein refers to data that is entered or stored into the data storage element before the array is used during an array process, i.e., data which is not collected during the processing or usage of the array and not related to a specific array process. In other words, data that would typically be entered before processing or using of the array, e.g., entered at the point of manufacture, etc., such data may include, but is not limited to, identification information, e.g., lot numbers, part numbers or other serial numbers, array layout information and other array descriptors, e.g., vendor identification information, date and/or time and/or location of manufacture and/or expiration and any other relevant array information. Static data is usually stored manually for example by manual input on a keyboard or via a computer mouse, or the like, but may be stored automatically as well.

### DETAILED DESCRIPTION OF THE INVENTION

Devices and methods for storing data related to at least one array are provided.

The subject devices are characterized by an array holder that holds a substrate having at least one array and a data storage element operatively coupled thereto and configured to receive and store data related to a held array. Stored data may include calibration data and/or the number of times the array is scanned. The data storage element may be configured to communicate with at least one external apparatus using a physical connection. In using the subject devices, a subject array holder holds a substrate having at least one array and data related to the at least one array is received and stored by the data storage element. The data storage element may communicate with at least one external apparatus using a physical connection. Also provided are kits for practicing the subject methods.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a device” includes a plurality of such devices and reference to “the array” includes reference to one or more arrays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Devices

As summarized above, the subject invention includes devices configured to collect, receive and store at least one of static and dynamic data, where such data can be easily and securely communicated, i.e., transferred to or received from, at least one external or remote apparatus or site such as an array assay apparatus, e.g., an array hybridization apparatus, a wash apparatus if separate from an array assay device, an incubator if separate from an array assay device, an array reader or scanner, a personal computer (“PC”), and the like. A subject array holder is capable of receiving and storing static and/or dynamic data in a data storage element associated with the array holder, where the data storage element is capable of communicating, i.e., transferring data to and/or receiving data from, at least one external or remote apparatus or site via a communication element associated with the array holder. The data storage element may be positioned in or on the array holder or in or on the substrate of the array. In further describing the subject devices, a description of a subject array holder capable of receiving and storing static and/or dynamic data substantially concurrently with the occurrence of an array process, event or condition is described first, followed by a description of a representative biopolymeric array for use with the subject array holders and which may also be capable of receiving and storing static and/or dynamic data concurrently with the occurrence of an array process, event or condition.

Array Holders

Generally, a substrate having at least one array is positioned in an array holder before subject to an array process, e.g., before it is transported, used in an array based assay such as an array hybridized assay, before it is read or scanned, etc. Thus, an array holder must be configured to hold an array substrate having at least one array, where such a configuration may vary depending on a variety of factors such as the type of array, the number of arrays, the configuration of the substrate, the particular function of the array holder, etc. For example, an array holder may function solely to retain an array, i.e., a simple housing unit that includes a chamber for holding the array (see for example U.S. Pat. No. 6,180,351 B1, the disclosure of which is herein incorporated by reference). Alternatively, the holder may be of a more complex nature and may include certain processing components. For example, an array holder may take the form of a hybridization apparatus or the like (see for example copending and commonly assigned application having U.S. Ser. No. 09/884,792, and entitled “Devices for Performing Array Hybridization Assays and Methods of Using the Same ”, filed Jun. 19, 2001 the disclosure of which is herein incorporated by reference.) Furthermore, an array holder may be configured to hold more than one substrate, i.e., a plurality of substrates may be held by an array holder. Still further, during a particular process, the array holder may be further associated with a device for holding the array holder or a plurality of such array holders in a fixed position, e.g., a carousel or the like. It is to be understood that all such array holders are contemplated by this invention. In other words, any array holder that is configured to hold at least one substrate having at least one array is contemplated by the subject invention.

Thus, the size and shape of the array holder will necessarily vary depending on a variety of factors, as described above. For example, the shape of an array holder may take any of a variety of shapes ranging from simple to complex. For example the holder may be of a substantially rectangular, square, circular, oblong, or oval shape, etc. Alternatively, as mentioned, the shape may be more complex such as a substantially irregular shape or the like. Likewise, the size of the array holder may also vary depending on a variety of factors. By way of example and not limitation, for those array holders configured to hold a single, rectangular substrate having one or more arrays, the length of the array holder will typically range from about 2 to about 200 mm, usually from about 50 to about 150 mm and more usually from about 80 to about 120 mm. The width of such an array holder will typically range from about 2 to about 200 mm, usually from about 40 to about 100 mm and more usually from about 40 to about 80 mm and the thickness of the array holder will typically range from about 0.5 to about 50 mm, usually from about 5 to about 25 mm and more usually from about 5 to about 15 mm. As mentioned above, in certain embodiments the array holder is circular in form, such as an optical storage disc or the like, where one or more arrays may be positioned around the circular compact disc, wherein the compact disc serves both to hold at least one array and receive and store data associated with the at least one array, i.e., serves also as a data storage element.

As described above, the subject array holders include, i.e., are operatively coupled to, a data storage element, where such a data storage element is capable of storing one or both of static data and dynamic data. By static data is meant data that is entered or stored into the data storage element before the array is used during an array process, i.e., data which is not collected during the processing or usage of the array. In other words, data that would typically be entered before processing or using of the array, e.g., entered at the point of manufacture, etc., such data may include, but is not limited to, identification information, e.g., lot numbers, part numbers or other serial numbers, array layout information and other array descriptors, e.g., vendor identification information, date of manufacture and/or expiration and any other relevant array information. Static data is usually stored, i.e., entered into the data storage element, manually for example by manual input on a keyboard or via a computer mouse, or the like, but may be stored automatically as well.

By dynamic data is meant data that is received and stored during the transport and processing of the array, where such data is received and stored substantially concurrently with the process or stored in “real time” in regards to the transport and process. In other words, dynamic data is data relating to array events, conditions, occurrences or processes to which the array is subject, where such data is stored or recorded at substantially the same time as the array events, conditions, occurrences or processes happen.

Examples of dynamic data include, but are not limited to, conditions or parameters of array transport or delivery or distribution (e.g., temperature, humidity, duration, etc., during the transport or delivery or distribution), conditions or parameters relating to an array assay such as an array hybridization assay and/or array scanning processes, e.g., temperature, humidity, gas concentration, duration of certain process steps, fluid volumes, fluid rates, number of times scanned, specific tag or label information, calibration information of ancillary apparatuses, etc. Dynamic data may also include data related to the conditions or parameters of the manufacturing process of the array, for example manufacturing conditions such as waveforms, waveform firing parameters, specific fluids used in the manufacturing process (e.g., manufacturer, concentration, lot number, part number, back pressures in the manufacturing vacuum system, etc.). Dynamic data may also include data related to quality control processes, where such data may be used to determine if specific array elements are faulty. Dynamic data may also include data relating to the identification of an external apparatus or instrument to which the device is associated, e.g., part number, lot number, manufacturer, etc.

Dynamic data may be received and stored manually such as with a keyboard or mouse, etc., or automatically, where the data will typically be received and stored in the data storage element automatically. That is, dynamic data may be entered and stored into a data storage element without any or without substantial human intervention, i.e., automatic dynamic data storage occurs without a human directly inputting data into the data storage element. A feature of the subject invention is that dynamic data is received and stored in the data storage element at the same time or substantially concurrently to the time that the process, condition, event is occurring. By substantially concurrently is meant, typically a time ranging from about 0 to about 300 seconds after the process or process parameter has occurred, usually from about 0 to about 100 seconds, and more usually from about 0 to about 5 seconds after the process, condition, event has occurred.

As described, the array holder is operatively coupled to a data storage element. In certain embodiments, more than one data storage element may be operatively coupled to a single array holder, as will be further described below. In many embodiments of the subject devices, the data storage element is positioned in or on the array holder itself. In certain other embodiments of the subject devices, the data storage element may be positioned in or on the substrate associated with at least one array. In any event, the data storage element is operatively coupled to a subject array holder, as mentioned above, so that data may be communicated or transferred between the data storage element and at least one external or remote apparatus or site via a communication element associated with the array holder, as will be further described below.

A feature of the subject data storage element is that it is capable of storing large amounts of data. In other words, the subject array holders or array substrates, i.e., the data storage elements of the holders or of the substrates, are capable of storing a greater amount of data than would be feasible to store on an array bar code. For example, due to size limitations as described above, an array bar code is usually confined to a storage capacity of less than about 100 bytes, whereas the subject array holders or arrays, and more specifically the data storage elements of the subject array holders or arrays, will typically have a storage capacity of about 100 bytes to about 500 megabytes or more, usually from about 250 bytes to about 15 megabytes and more usually from about 0.125 megabytes to about 4 megabytes.

Another feature of the subject data storage element is that it is operatively coupled to at least one parameter measuring sensor for measuring and/or monitoring certain parameters or conditions associated with at least one array. For example, such a parameter measuring sensor may measure parameters or conditions to which an array is subjected, e.g., temperature, humidity, etc., during certain array events or processes, the number of times an array has been scanned. Other parameters which may be measured include, but are not limited to, the type and/or volume of reagents used during an array process and the rate at which fluid is dispensed, gas concentrations, calibration information related to ancillary or external apparatuses used with the array holder, e.g., the parameter or specification a reagent was set to be dispensed when calibrated, date and/or time and/or location of calibration, operator identification, duration of certain process steps, etc., the type, lot number, part number, manufacturer of reporter tag or label used during the array assay process, e.g., type of dye used for the fluorescent label, the identification of a plurality of arrays on a single substrate, certain conditions or parameters of array transport or delivery or distribution, e.g., duration of transport, temperature, humidity, etc., conditions or parameters relating to array assays, e.g., hybridization assays and/or array scanning processes, such as the initial and terminal incubation temperatures to which the array holder was subjected, gas concentrations, identification of ancillary devices (e.g., lot number, part number, manufacturer, etc.), the duration of the incubation(s), the initial and terminal temperatures of the first and any subsequent wash cycles, the durations of each wash cycle, the stringency of each wash fluid, the duration of the drying step, etc., the data related to quality control processes, and the like. Such an operative coupling of the data storage element to at least one measuring sensor provides an easy way to automatically collect data, e.g., static and/or dynamic data, and store it in the data storage element, usually automatically as well.

The parameter measuring sensor may be any convenient measuring sensor, including, but not limited to, temperature sensors such as thermistors, and the like, humidity sensors, volume sensors, gas concentration sensors, time or durational sensors, optical sensors, and the like. The parameter measuring sensor may be integrated into the array holder, the substrate the array is positioned on or may be a part of or integrated with a separate component that is operatively coupled with the data storage element of the array holder or the substrate, e.g., it may be a part of an array assay apparatus such as a hybridization apparatus and/or wash apparatus and/or an array scanning apparatus, and the like. In certain embodiments, the substrate the array is deposited on may be an integrated circuit with a passivated surface upon which the array is positioned and one or more parameter measuring sensor and/or the data storage element may be included in the integrated circuit.

Accordingly, the subject devices also include, i.e., are operatively coupled to, one or more microprocessors, where such one or more microprocessor are under the control of a software program and thus are suitably programmed to execute all of the steps or functions required of them, or any hardware or software combination that will perform such required functions. In one instance, the microprocessor receives information from the measuring sensor. For example, a microprocessor receives voltage information from the one or more measuring sensors, where such voltage information is related to a particular process parameter or quantity or magnitude thereof, and digitizes the information to be stored in the data storage element. In other instances, a microprocessor may be capable of turning on and/or off status indicating LEDs associated with a display panel of a processing apparatus, such as when the array holder is operatively associated with or disassociated from the apparatus, or turning on and/or off certain valves or the like of an array processing apparatus such as an array hybridization apparatus and/or wash apparatus or the like to expose the array holder, i.e., the array in the array holder, to different chemical or environmental conditions. The one or more microprocessors may carry out all the steps necessary to process and/or encrypt data, e.g., before the data is stored in the data storage element or before data is transferred from the data storage element.

The subject devices are thus configured to communicate with external or remote apparatuses or sites. In other words, an array holder may be configured to be operatively coupled to an external device such as one or more of any suitable array assay apparatus, e.g., an array hybridization apparatus and/or wash apparatus such as that which is disclosed in copending and commonly assigned application having U.S. Ser. No. 09/919,073 and entitled “Sample Processing Apparatus and Methods”, filed Jul. 30, 2001; and U.S. Pat. Nos. 6,238,910; 5,958,760; and 6,114,122, the disclosures of which is herein incorporated by reference. Other suitable external or remote apparatuses may include an array reading or scanning apparatus such as any suitable apparatus for interrogating an array, such as one which can read the location and intensity of bound labels or tags, e.g., fluorescently or radioactively bound labels (for example an array reader such as a MICROARRAY scanner available from Agilent Technologies of Palo Alto, Calif.), a PC, an incubator and the like. The one or more external or remote apparatuses to which the subject devices may communicate, i.e., receive and/or collect information from the subject device, where such received information may be used by the external apparatus to control particular functions of the external apparatus, assist in the analysis of data regarding an array, etc. The one or more external or remote apparatuses may also transfer data to a subject device, e.g., calibration information, identification information (e.g., lot number, part number, manufacturer, etc.) related to the external apparatus.

More specifically, the data storage element of the array holder is configured to communicate, i.e., transfer data to and/or receive data from at least one ancillary external or remote apparatus or site or instrument via a communication element, where such a communication element is positioned in or on an array holder. The communication may be accomplished automatically upon coupling of the communication element with the external apparatus, e.g., once a physical connection is provided. The communication element is capable of enabling automatic communication between the array holder, particularly the data storage element of the array holder or the data storage element of an array substrate which is associated with an array holder, and at least one external or remote apparatus. In general, the communication element employed by the subject invention is typically one which is reliable, does not require a separate power source such as a battery or the like, does not require an antenna, is minimally susceptible to interference from external apparatuses, e.g., is minimally susceptible to interfering electromagnetic radiation and the like from nearby apparatuses including nearby subject devices, and is appropriately secure, i.e., data transferred thereby is not easily intercepted or read by unintended parties.

Of particular interest are those communication elements that employ a physical or hard connection, i.e., communication elements that require physical contact or coupling between the communication element and the one or more external apparatuses with which communication is desired, for example a suitable cable connector or suitable serial port or dock that can be operatively associated with a corresponding cable or pin element or the like. However, other communication elements are contemplated by this invention as well such as a wireless system such as a wireless system that communicates via radiofrequency and the like. Representative communication elements include, but are not limited to, Serial Peripheral Interface (“SPI”), MICROWIRE, I²C, +5 V serial, 1-Wire® interface, General Purpose Interface Bus (“GPIB”), memory mapped I/O, RS-232, Peripheral Component Interconnect (“PCI”), Universal Serial Bus (“USB”), ETHERNET® and infrared communications such as Infrared Data Associations (“IrDATm™”). Furthermore, the communication element may be one which is configured to communicate via telephone network, satellite network, Wide Area Network (“WAN”), and the like.

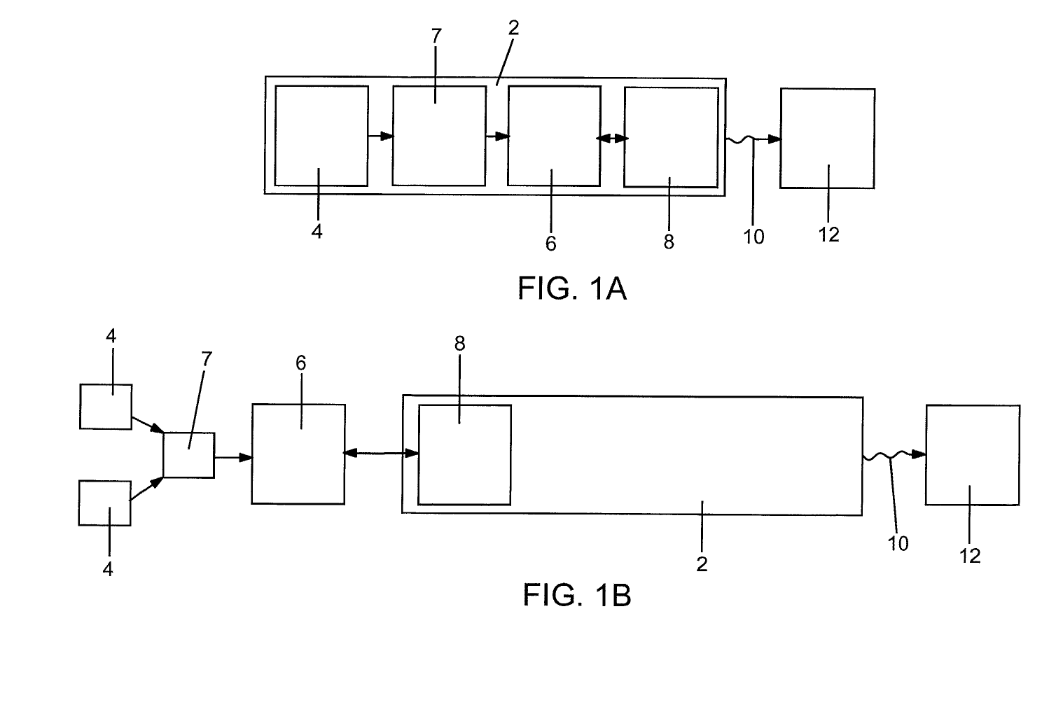

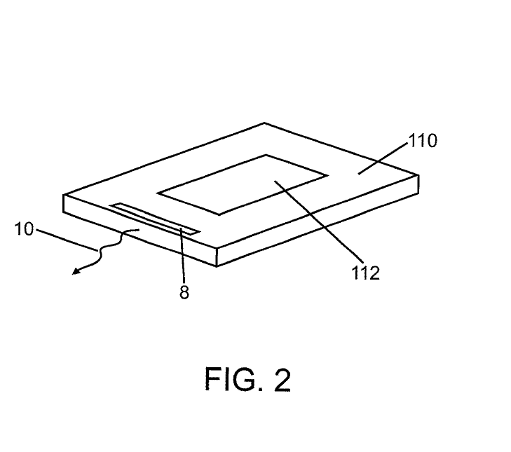

Referring now to the Figures where like numerals represent like features or elements, FIG. A shows an exemplary embodiment of a subject device. Accordingly, the device shown in FIG. 1A is capable of receiving and storing static data, as well as receiving and storing dynamic data, i.e., capable of receiving and storing data related to and substantially concurrently with the occurrence of the process, event or condition to which an array is subjected, usually automatically. The device of FIG. 1A thus includes array holder 2, where such an array holder includes (1) at least one measuring sensor 4, such as an optical sensor and/or a temperature and/or a humidity sensor, etc. (where more than one may be included), (2) a signal conversion element 7 such as an analog to digital signal converter or the like, (3) at least one microprocessor, herein shown as a single microprocessor 6, and (4) a data storage element 8. The array holder 2 is capable of communicating with one or more external or remote apparatuses 12 via communication element 10, where external apparatus 12 may include one or more of an array assay device, e.g., a hybridization apparatus, a wash apparatus, an incubator, an array scanning apparatus, a PC, and the like. In this particular embodiment, the data storage element 8 is positioned in or on the array holder 2. However, as described above, the data storage element 8 may be positioned in or on a substrate having at least one array, where the substrate is associated with the array holder 2 having a communication element 10, such as will be described in reference to FIG. 2 below. In the embodiment shown in FIG. 1A, the array holder 2 includes the measuring sensor(s) 4, a signal converter 7, microprocessor 6 and a data storage element 8. However, in certain other embodiments of the subject invention, one or more of the above features or components are positioned external to the array holder, yet operatively associated with the data storage element 8 of the array holder. FIG. 1B shows such an embodiment where the data storage element 8 is positioned or located on or in the array holder 2 and the measuring sensor(s) 4, signal converter 7 and microprocessor 6 are external to the array holder 2, yet operatively associated with the data storage element 8.

As described above, the data storage element may be positioned in or on a substrate having at least one array where the substrate, and thus the at least one array, is associated with an array holder having a communication element. Accordingly, FIG. 2 shows an exemplary embodiment of a substrate 110 carrying one or more arrays 112, where such substrate 110 and one or more arrays 112 will be described in greater detail below. It will be understood that substrate 110 and any embodiments thereof may use any number of desired arrays 112. The array(s) 112, and more particularly the substrate 110 associated with the array(s) 112, includes data storage element 8. It will be understood that while this particular embodiment includes one data storage element 8, a plurality of data storage elements may be included such that each data storage element may be associated with a single, respective array. Substrate 110 further includes a communication element 10 for communicating between the substrate 110 and an array holder or other external apparatus or component, such as a measuring element, microprocessor, signal converter, and the like, as described above. For example, substrate 110 may be placed in an array holder such as array holder 2 of FIGS. 1A and 1B and thus operatively associated with the measuring element, signal converter and microprocessor associated with the array holder into which it is placed. In certain embodiments, in addition to the data storage element 8 shown in FIG. 2, the substrate 110 associated with array 112 may include one or more of the following: measuring sensor(s), signal converter and microprocessor (not shown). However, as described above, one or more of the above mentioned elements may be external to the array substrate 110, i.e., in or on the array holder into which the substrate 110 is placed or external to both the array substrate 110 and array holder.

As described above, usually the subject devices do not include or require a separate power source for supplying power to one or more components of the device. In many embodiments, power is supplied to the device via an ancillary or external apparatus to which the device is associated, such as a hybridization apparatus and/or wash apparatus and/or an array reader or scanner, etc., as described above. However, in certain other embodiments, the device includes its own power source such as a battery or the like. As is apparent, in those instances where the device includes a power source separate from a power source of an external device, the device is capable of functioning, e.g., collecting, receiving and storing data, when not associated with an ancillary device (i.e., when not physically connected thereto), for example during transport from a manufacturing site, etc.

Representative Biopolymeric Arrays

As mentioned above, the devices of the subject invention are used with arrays and more specifically biopolymeric arrays. Such biopolymeric arrays find use in a variety of applications, including gene expression analysis, drug screening, nucleic acid sequencing, mutation analysis, and the like. These biopolymeric arrays include a plurality of ligands or molecules or probes (i.e., binding agents) deposited onto the surface of a substrate in the form of an “array” or pattern.

The subject biopolymeric arrays include at least two distinct polymers that differ by monomeric sequence attached to different and known locations on the substrate surface. Each distinct polymeric sequence of the array is typically present as a composition of multiple copies of the polymer on a substrate surface, e.g. as a spot on the surface of the substrate. The number of distinct polymeric sequences, and hence spots or similar structures, present on the array may vary, but is generally at least 2, usually at least 5 and more usually at least 10, where the number of different spots on the array may be as a high as 50, 100, 500, 1000, 10,000 or higher, depending on the intended use of the array. The spots of distinct polymers present on the array surface are generally present as a pattern, where the pattern may be in the form of organized rows and columns of spots, e.g. a grid of spots, across the substrate surface, a series of curvilinear rows across the substrate surface, e.g. a series of concentric circles or semi-circles of spots, and the like. The density of spots present on the array surface may vary, but will generally be at least about 10 and usually at least about 100 spots/cm², where the density may be as high as 10⁶ or higher.

In the broadest sense, the arrays of the subject invention are arrays of polymeric or biopolymeric ligands or molecules, i.e., binding agents, where the polymeric binding agents may be any of: peptides, proteins, nucleic acids, polysaccharides, synthetic mimetics of such biopolymeric binding agents, etc. In many embodiments of interest, the arrays are arrays of nucleic acids, including oligonlucleotides, polynucleotides, cDNAs, mRNAs, synthetic mimetics thereof, and the like.

The arrays may be produced using any convenient protocol. Various methods for forming arrays from pre-formed probes, or methods for generating the array using synthesis techniques to produce the probes in situ, are generally known in the art. See, for example, U.S. Pat. Nos. 5,700,637; 5,143,854 and Fodor, et al. (1991) Science 251:767-777, the disclosures of which are incorporated herein by reference and PCT International Publication No. WO 92/10092. For example, probes can either be synthesized directly on the solid support or substrate to be used in the array assay or attached to the substrate after they are made.

A variety of solid supports or substrates may be used, upon which an array may be positioned. In certain embodiments, a plurality of arrays may be stably associated with one substrate. For example, a plurality of arrays may be stably associated with one substrate, where the arrays are spatially separated from some or all of the other arrays associated with the substrate.

The substrate may be selected from a wide variety of materials including, but not limited to, natural polymeric materials, particularly cellulosic materials and materials derived from cellulose, such as fiber containing papers, e.g., filter paper, chromatographic paper, etc., synthetic or modified naturally occurring polymers, such as nitrocellulose, cellulose acetate, poly (vinyl chloride), polyamides, polyacrylamide, polyacrylate, polymethacrylate, polyesters, polyolefins, polyethylene, polytetrafluoro-ethylene, polypropylene, poly (4-methylbutene), polystyrene, poly(ethylene terephthalate), nylon, poly(vinyl butyrate), cross linked dextran, agarose, etc.; either used by themselves or in conjunction with other materials; fused silica (e.g., glass), bioglass, silicon chips, ceramics, metals, and the like. For example, substrates may include polystyrene, to which short oligophosphodiesters, e.g., oligonucleotides ranging from about 5 to about 50 nucleotides in length, may readily be covalently attached (Letsinger et al. (1975) Nucl. Acids Res. 2:773-786), as well as polyacrylamide (Gait et al. (1982) Nucl. Acids Res. 10:6243-6254), silica (Caruthers et al. (1980) Tetrahedron Letters 21:719-722), and controlled-pore glass (Sproat et al. (1983) Tetrahedron Letters 24:5771-5774). Additionally, the substrate can be hydrophilic or capable of being rendered hydrophilic.

Suitable substrates may exist, for example, as sheets, tubing, spheres, containers, pads, slices, films, plates, slides, strips, disks, etc. The substrate is usually flat, but may take on alternative surface configurations. The substrate can be a flat glass substrate, such as a conventional microscope glass slide, a cover slip and the like. Common substrates used for the arrays of probes are surface-derivatized glass or silica, or polymer membrane surfaces, as described in Maskos, U. et al., Nucleic Acids Res, 1992, 20:1679-84 and Southern, E. M. et al., Nucleic acids Res, 1994, 22:1368-73.

Immobilization of the probe to a suitable substrate may be performed using conventional techniques. See, e.g., Letsinger et al. (1975) Nucl Acids Res. 2:773-786; Pease, A. C. et al., Proc. Nat. Acad. Sci. USA, 1994, 91:5022-5026. and “Oligonucleotide Synthesis, a Practical Approach,” Gait, M. J. (ed.), Oxford, England: IRL Press (1984). The surface of a substrate may be treated with an organosilane coupling agent to functionalize the surface. See, e.g., Arkins, “Silane Coupling Agent Chemistry,” Petrarch Systems Register and Review, Eds. Anderson et al. (1987).

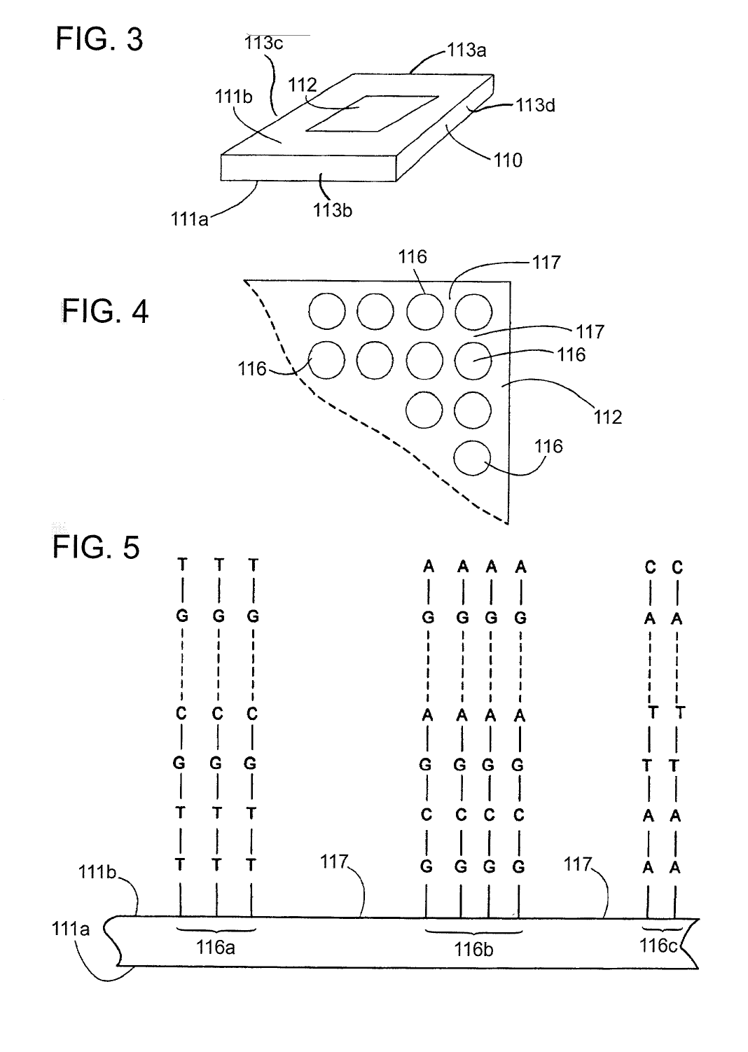

Referring first to FIGS. 3-5, typically biopolymeric arrays of the present invention use a contiguous planar substrate 110 carrying an array 112 disposed on a rear surface 111b of substrate 110. It will be appreciated though, that more than one array (any of which are the same or different) may be present on rear surface 11b, with or without spacing between such arrays. The one or more arrays 112 cover only a portion of the rear surface 1111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as described above, and any subject array assay apparatus or scanner used with it adapted accordingly.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

As noted above, the arrays of the subject invention find use in a variety applications, where such applications are generally analyte detection applications, as mentioned above, in which the presence of a particular analyte in a given sample is detected at least qualitatively, if not quantitatively. Protocols or techniques for carrying out such array assays are well known to those of skill in the art and need not be described in great detail here. Generally, a sample suspected of comprising the analyte of interest is contacted with an array under conditions sufficient for the analyte to bind to its respective binding pair member that is present on the array. Thus, if the analyte of interest is present in the sample, it binds to the array at the site of its complementary binding member, forming a complex on the array surface. The presence of this binding complex is then detected, e.g., through use of a signal production system, e.g., fluorescent label present on the analyte, etc. The presence of the analyte in the sample is then deduced from the detection of binding complexes on the substrate surface.

As mentioned above, the biopolymeric arrays may be used in a variety of array based assays, where hybridization reactions will be used herein for exemplary purposes only, and is not intended to limit the scope of the invention. In hybridization assays, a sample of target analyte such as target nucleic acids is first prepared, where preparation may include labeling of the target nucleic acids with a label, e.g., with a member of signal producing system and the sample is then contacted with the array under hybridization conditions, whereby complexes are formed between target analytes such as nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patent applications describing methods of using arrays in various applications include: WO 95/21265; WO 96/31622; WO 97/10365; WO 97/27317; EP 373 203; and EP 785 280 and U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of the U.S. Patents which are herein incorporated by reference.

In certain embodiments, the subject invention include a step of transmitting data from at least one of the detecting and deriving steps, as described above, to a remote location. By “remote location” is meant a location other than the location at which the array is present and the array assay occurs For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

### Systems

Also provided by the subject invention are systems for use with the subject devices, where such systems include, but are not limited to, at least one substrate having at least one array and an array holder operatively coupled to at least one data storage element. The system may further include at least one of an external or remote apparatus, a microprocessor, a signal converter and a sensor, as described above.

### Methods of Use

As summarized above, the subject invention also provides methods for collecting, receiving and storing data related to an array, where such data includes at least one of static and dynamic data, where any one or all of the collection, receipt and storage of the data storage element may be accomplished automatically. In certain embodiments of the subject methods, data may be collected from and/or transferred to at least one external or remote apparatus or site automatically. Specifically, a substrate having at least one array is held by an array holder and is subjected to one or more processes, events or conditions, such as an array assay, e.g., an array hybridization assay, array scanning or reading, transport and the like. Information related to the one or more array processes, events or conditions is received and stored by a data storage element associated with the array holder (on or in the holder and/or on or in the substrate of the array), in certain embodiments while the process, event or condition of interest is occurring or substantially close to or near the time the process is occurring, i.e., the data is received and stored in real time relative to the time of the occurrence of the process, event or condition.

Accordingly, the first step in the subject methods is to provide an array holder configured to receive at least one array and which is operatively coupled to a data storage element capable of receiving data and storing one or both of static and dynamic data, such as the array holder described above. The array holder, as mentioned above, is configured to operatively associate with at least one external device to provide communication therebetween, i.e., transfer data to and/or receive data from at least one external or remote apparatus, where such a configuration typically includes a physical or hard connection.

In particular, data ranging from about 100 bytes to about 500 megabytes or more, usually from about 250 bytes to about 15 megabytes and more usually from about 0.125 megabytes to about 4 megabytes of data may be received and subsequently stored by the data storage element. As described above, the data storage element may be on or in the array holder itself and/or may be on or in the substrate associated with the array and receipt and storage may be accomplished automatically.

After the provision of a suitable array holder, a substrate having at least one array such as an array of the type described above, i.e., a biopolymeric array, is placed in the array holder. At this point or any point thereto, data may be stored in the data storage element operatively coupled to the array holder, i.e., static data may be received by and stored in the data storage element, as described above. For example, information such as lot numbers, part numbers or other serial numbers, array layout information, and other array descriptors, e.g., vendor identification information, date and/or time and/or location of manufacture or expiration, and other relevant array information, may be received and stored, where such static data is typically entered into the data storage element manually by a human, e.g., inputted using a keyboard, computer mouse, or the like.

The next step is to subject the array, i.e., subject the array that is held in the suitable array holder, to one or more array processes. As described above, the array process, event or condition may include one or a variety of array processes including those related to transport or distribution, those related to an array assay such as an array hybridization assay, those related to reading or scanning of an array, etc. and information is collected, received and stored. Specifically information may include, but is not limited to, humidity, temperature, amount of time for assay completion, type, volume and rate of reagent used, type of reporter tag used, calibration information related to external apparatuses, the number of times scanned, external apparatus identification information, etc. Of particular interest is the collection of data regarding the number of times the array has been scanned and calibration data associated with external apparatuses such as an array assay device, e.g., an array hybridization chamber and an array reader, as will be described in greater detail below.

Thus, in accordance with the subject invention, parameters or conditions associated with one or more array processes are measured and/or monitored and then received and stored by the data storage element operatively coupled to the array holder substantially concurrently with the occurrence of the process or particular process parameter of interest, i.e., dynamic data is received and stored by the data storage element, where a large amount of data may be stored, as described above. By substantially concurrently is meant typically a time ranging from about 0 to about 300 seconds after the process or process parameter has occurred, usually from about 0 to about 100 seconds, and more usually from about 0 to about 5 seconds after the process or process parameter has occurred.

As mentioned above, the subject methods also include the steps of measuring and/or monitoring, usually automatically, certain parameters or conditions associated with at least one array, during certain array events or processes, such as transport, array assays, e.g., hybridization assays, scanning processes, etc. Such parameters or conditions may include, but are not limited to, temperature data, humidity data, identification information relating to an external apparatus to which a subject device is associated, e.g., lot number, part number, manufacturer, etc., the number of times an array has been scanned, the rate of fluid dispensation, gas concentrations, the duration of incubation(s), the duration of one or more wash cycles, the stringency of each wash fluid, the duration of the drying step, calibration information related to ancillary or external apparatuses used with the array holder, the type and/or volume of reagents used during an array process, the type of reporter tag or label used during the array assay process, e.g., type and/or lot number and/or part number and/or manufacturer of dye used for the fluorescent label, the identification of a plurality of arrays on a single substrate, certain conditions or parameters of array transport or delivery or distribution, data related to quality control processes, and the like. The measured and/or monitored data is then usually digitized and stored in the data storage element.

As described above, such dynamic data may be received and stored manually, but is typically received and stored automatically. In other words, data related to parameters or conditions associated with one or more array process is measured and/or monitored, and received and stored without any or without substantial human intervention. More specifically, the dynamic data need not be manually inputted into the data storage element, but is instead done so automatically, substantially during the occurrence of the array process or event.

As mentioned above, of particular interest is the collection and storage of dynamic data related to calibration data of an ancillary or external apparatus that is operatively associated with a subject array holder, e.g., an array assay device such as an array hybridization apparatus and/or wash apparatus and/or incubator and/or an array reader or scanner, etc. Accordingly, calibration data associated with the external apparatus or any component thereof is received and stored in the data storage element, where examples of calibration data include, but are not limited to, the date and/or time and/or location of calibration, the actual calibrated setting, the ideal calibration setting(s), etc. For example, in regards to calibration data of an array scanner, calibration data may include the speed at which the array stage was set when calibrated, the focus depth to which the laser was set when calibrated, the position or angle that a scanner mirror was set when calibrated, certain light source and/or detector calibration data, the date and/or time and/or location of calibration, the identification of the operator who performed the calibration, etc.

Likewise, as mentioned above, calibration data relating to an array assay device such as a hybridization apparatus and/or wash apparatus may also be collected and stored, where such calibration data may include, but is not limited to, humidity calibration data, the rate at which fluid was calibrated or set to be dispensed when calibrated, the value that the initial and terminal incubation temperatures were set to when calibrated, the stringency to which one or more wash fluids was set when calibrated, the duration of time that an incubation period was set to when calibrated, the concentration to which a gas was set during calibration, the values to which the initial and terminal temperatures of the first and any subsequent was cycles were set when calibrated, the durations of time to which ach wash cycle was set during calibration, the duration of time to which each of the drying steps was set during calibration, the identification of the operator who performed the calibration, the date and/or time and/or location of calibration, etc.

Any or all of the stored data may be organized into separate or discrete areas in the data storage element. For example, data may be stored in areas that are generally or broadly accessible and/or stored in areas that have limited accessibility, e.g., the areas are protected and accessible only if a password is provided or the like. Furthermore, the data may be stored in a variety of formats, including, but not limited to, raw, processed, encrypted and decrypted formats. In certain embodiments of the subject invention, certain data may be stored in generally accessible areas and certain other data may be stored in limited access areas, where some or all of the data stored in either or both of the generally accessible areas and/or limited access areas may be raw and/or processed and/or encrypted and/or decrypted.

In addition to receiving and storing data, the subject methods also include communicating static and/or dynamic data between the subject device and at least one external or remote apparatus such as a hybridization apparatus and/or wash apparatus, scanner, PC, and the like, i.e., data may be easily or readily transferred to or received from an external apparatus. In certain embodiments, the data transferred is used in a subsequent or downstream process. For example, information regarding a particular reagent such as a hybridization reagent may be transferred from a data storage element to an array scanner for use in one or more subsequent scanning processes, e.g., if the particular reagent lot number is known to produce a particular background signal, the information can be incorporated into the reading of the array to correct for such background signal. In another example, data relating to extreme transport conditions may be transferred from a data storage element to an array assay device such as a hybridization apparatus and/or wash apparatus to be used in a subsequent array assay process, e.g., an array hybridization assay, to evaluate the binding efficiency of a binding pair and/or transferred to an array scanner to be used in a scanning process to determine the origin of irregular or erroneous array readings. In another example, calibration data relating to an external apparatus may be transferred to the data storage element and later transferred from the data storage element to another external apparatus, where such calibration data may be used to determine if an external apparatus is functioning within specification and/or used to adjust other components to a particular calibrated setting, and/or to evaluate array reading results, e.g., to determine the origin of irregular or erroneous results, etc. The data storage element may also communicate with a personal computer (“PC”), where such a PC may generate relevant data regarding the communicated data and thus regarding the array, i.e., the processing of the array and/or transfer data to the data storage element.

Accordingly, to communicate with an external or remote apparatus, a variety of mechanisms may be employed, as noted above. In certain embodiments, a physical connection or hard connection is employed to operatively associate the data storage element with an external device so that communication between the two may be achieved. In such an embodiment, at anytime prior to the transfer of stored data, the array holder is physically connected to the external apparatus. Once a suitable physical contact is made, data may be transferred between the data storage element and the external apparatus. As described above, other mechanisms may be used in addition to or instead of a mechanism that requires a physical connection, e.g., a wireless system.

In certain embodiments, data may be password protected and thus the data may only be transferred to or from an external apparatus if a password is provided. The data may be transferred in an encrypted or decrypted format or may be transferred in a raw or processed format.

The subject methods may further include automatically activating and/or deactivating various components of one or more external or remote apparatuses during the processing of an array. In other words, a subject array holder may turn on and/or off one or more external or remote apparatuses associated therewith or a component of such associated apparatuses may be turned on and/or off, usually during the processing of the array. In one aspect, one or more external apparatuses or a component thereof may be under the control of an array holder, where such an array holder may be responsible for activating and/or de-activating the external apparatus or a component thereof. For example, once associated with an array holder, an array holder may activate and/or de-activate, i.e., turn on and/or off, status indicator LEDs associated with the external apparatus. More specifically, an array holder may be associated with an external device such as by a suitable communication element, as described above, where such association then turns on an “ON” status LED of the apparatus. Rather, a microprocessor associated with the array holder carries out the steps necessary to turn on the status LED. Likewise, once the array holder is removed from the apparatus, e.g., once the physical connection between the array holder and the external apparatus is broken, the status LED is de-activated to an “OFF” status. Similarly, the array holder may activate and/or de-activate valves or the like associated with one or more external apparatuses, where such valves may be opened to expose the array to one or more reagents and thus closed to stop the exposure and/or may activate or deactivate heating elements or set temperatures thereof.

### Methods For Processing an Array

Also provided by the subject invention are methods for processing an array. In general, the subject methods include performing an array process and receiving and storing data related to the process, wherein the data is received and stored substantially concurrently with the process. As described above, an array process includes a variety of processes, events or conditions to which the array is subjected and may include, but is not limited to, transport or distribution, storage, an array assay such as an array hybridization assay, array reading or scanning, e.g., the number of times the array is scanned, calibration data, etc.

Thus, in the subject methods for processing an array, the first step is to provide an array holder configured to receive at least one array and which is operatively coupled to a data storage element capable of receiving and storing at least one of static and dynamic data. In particular, the data storage element is capable of storing about 100 bytes to about 500 megabytes of data or more, usually from about 250 bytes to about 15 megabytes and more usually from about 0.125 megabytes to about 4 megabytes of data may be stored by the data storage element, where data may be stored automatically. As mentioned above, the data storage element may be positioned on or in the array holder or on or in the substrate associated with at least one array.

An array is then placed in the array holder, such as an array of the type described above, i.e., a biopolymeric array. At this point or any point thereto, data may be received and stored by the data storage element operatively coupled to the array holder, i.e., static data, as described above, may be stored in the data storage element. For example, information such as lot numbers, part numbers or other serial numbers, array layout information, and other array descriptors, e.g., vendor identification information, date of manufacture or expiration, and other relevant array information, may be stored.

The next step is to subject the array, i.e., the array that is retained in a suitable array holder, to one or more array processes. As described above, the array processes may include a variety of processes including transport or distribution or storage of an array, performance of an array assay such as an array hybridization assay, reading or scanning an array, etc.

For example, the array may be subjected to a transportation process, e.g., from the manufacturing site to an end user site such as a laboratory or the like and information related to the transport may be collected and stored. Furthermore, the array may be subject to an array assay such as an array hybridization assay, where a sample of target molecules or the like is contacted with the array under suitable conditions, whereby complexes are formed between target molecules that are complementary to probe sequences attached to the array surface. Specific array assays of interest which may be practiced using the subject arrays include a variety of binding assays. For example, a variety of array hybridization assays may be practiced with the subject invention and include, but are not limited to: gene discovery assays, differential gene expression analysis assays, nucleic acid sequencing assays, and the like. Still further, the array that has been contacted with a sample containing target molecules or members of a specific binding pair, may then be subjected to a scanning or reading process, where such a process includes scanning the processed array, usually with an optical source or the like. Representative scanners include that perform such processes include, but are not limited to, commercially available scanners such a MICROARRAY scanner available from Agilent Technologies of Palo Alto, Calif., or scanners such as those disclosed in U.S. Pat. Nos. 5,585,639 and 6,258,593, the disclosures of which are herein incorporated by reference, where the presence of the bound complexes is then detected.

During any one of at least the above mentioned processes, data is received and stored substantially concurrently with the process. In other words, parameters associated with one or more array processes, events or conditions are received and stored by the data storage element operatively coupled to the array holder substantially concurrently with the occurrence of the process or particular process parameter of interest, i.e., dynamic data is received and stored by the data storage element, where a large amount of data may be stored, as described above. By substantially concurrently is meant typically a time ranging from about 0 to about 300 seconds after the process or process parameter has occurred, usually from about 0 to about 100 seconds, and more usually from about 0 to about 5 seconds after the process or process parameter has occurred. Such parameters include, but are not limited to, process temperature, humidity, time, reagent volumes, number of times scanned, calibration data, and the like.

Such dynamic data may be received and stored manually, but is typically received and stored automatically. In other words, data related to parameters or conditions associated with one or more array processes is received and stored without substantial human intervention. More specifically, the dynamic data need not be manually collected, inputted and stored, but is instead done so automatically. The data may be stored in segregated areas, e.g., protected or secured areas and may also be stored encrypted.

The subject methods also include the steps of measuring and/or monitoring, usually automatically, certain parameters or conditions associated with at least one array, e.g., temperature, humidity, etc., during certain array events or processes, the number of times an array has been scanned, calibration information related to ancillary or external apparatuses used with the array holder, the type and/or volume of reagents used during an array process, the type of reporter tag or label used during the array assay process, e.g., type of dye used for the fluorescent label, the identification of a plurality of arrays on a single substrate, certain conditions or parameters of array transport or delivery or distribution, certain conditions or parameters relating to array assays, e.g., hybridization assays and/or array scanning processes, data related to quality control processes, and the like. The measured and/or monitored data is then usually digitized and stored in the data storage element.

Furthermore, data may be communicated between a subject device and at least one external or remote apparatus such as an array assay apparatus, e.g., an array hybridization apparatus, array reader or scanner, PC, and the like, as described above. The data may be transferred in an encrypted format or may be transferred in a decrypted format.

The subject methods may further include activating and/or de-activating various components of one or more external or remote apparatuses during the processing of an array, as described above.

### Kits

Finally, kits for use in storing data related to an array are provided. The subject kits may include one or more subject array holders, i.e., array holders operatively coupled to a data storage element or a plurality of data storage elements.

Typically, a plurality of subject array holders is included. The kits may further include a processing apparatus such as an array assay device, e.g., a hybridization apparatus, an array reader or scanner, or the like. The subject kits may also include one or more arrays, where the arrays may or may not include an operatively coupled data storage element. The kits may further include one or more additional components necessary for carrying out an analyte detection assay, such as sample preparation reagents, buffers, labels, and the like. As such, the kits may include one or more containers such as vials or bottles, with each container containing a separate component for the array assay, and reagents for carrying out an array assay such as a nucleic acid hybridization assay or the like. The kit may also include a denaturation reagent for denaturing the analyte, buffers, e.g., hybridization buffers, wash mediums, enzyme substrates, reagents for generating a labeled target sample such as a labeled target nucleic acid sample, negative and positive controls and written instructions for using the subject array assay devices for carrying out an array based assay and/or for collecting, storing and transferring data related to at least one array. The instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc.

It is evident from the above results and discussions that the above described invention provides a simple way to store both static and dynamic data, and in particular provides a simple way to store large amounts of such data. The above described invention provides for a number of advantages, including the abilities to receive and store both static and dynamic data manually or automatically, automatically collect dynamic information over time, store a large amount of such data, i.e., one or more megabytes, easily communicate with external apparatuses and/or sites and automatically activate and/or deactivate external apparatuses or components thereof. The subject invention also provides a way to store data in protected or secure areas and also provides a way to store encrypted, as well as decrypted data. Furthermore, the subject invention includes ways to transfer data between a subject device and an external apparatus in a secure manner with little risk of interference, such as electromagnetic interference from surrounding apparatuses and/or other subject devices. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A device for storing data related to at least one array, said device comprising:
an array holder for holding a substrate having at least one array; and
a data storage element operatively coupled to said array holder and configured to receive and store data related to said at least one array, wherein said data storage element is capable of communicating with at least one external apparatus using a physical connection.

2. The device according to claim 1, wherein said data storage element is capable of automatically storing data.

3. The device according to claim 1, wherein said data storage element is capable of automatically communicating with at least one external apparatus.

4. The device according to claim 1, wherein said external apparatus comprises at least one of an array assay apparatus, a wash apparatus, an incubator, an array scanner and a personal computer.

5. The device according to claim 1, wherein said physical connection comprises a communication element selected from the group consisting of Serial Peripheral Interface (“SPI”), MICROWIRE, I²C, +5 V serial, 1-Wire® interface, General Purpose Interface Bus (“GPIB”), RS-232, Peripheral Component Interconnect (“PCI”), memory mapped I/O and Universal Serial Bus (“USB”).

6. The device according to claim 1, where said data is encrypted.

7. The device according to claim 1, wherein said data is password protected.

8. The device according to claim 1, wherein said data storage element is capable of storing from about 100 bytes to about 500 megabytes or more of data.

9. The device according to claim 1, wherein said data is at least one of dynamic data and static data.

10. The device according to claim 1, wherein said data is at least one of calibration data, array assay information and the number of times said at least one array is scanned.

11. The device according to claim 1, further comprising a sensor for measuring or monitoring a condition, event or process to which said array is subjected.

12. A device for storing data related to at least one array, said device comprising:
an array holder for holding a substrate having at least one array; and
a data storage element operatively coupled to said array holder and configured to receive and store data related to said at least one array, wherein said data comprises at least one of calibration data, array assay information and the number of times said at least one array is scanned.

13. The device according to claim 12, wherein said calibration data comprises data related to at least one of array stage speed, focus depth, scanner mirror position, light source calibration, detector calibration, humidity, temperature, fluid volume, rate of fluid dispensation, fluid stringency, duration of incubation, gas concentration, duration of wash cycle, duration of drying, date of calibration, time of calibration, location of calibration and operator identification.

14. The device according to claim 12, wherein said data storage element is capable of communicating with at least one external apparatus.

15. The device according to claim 14, wherein said data storage element is capable of automatically communicating with said at least one external apparatus.

16. The device according to claim 14, wherein said device comprises at least one of a physical connection and a wireless system.

17. The device according to claim 16, wherein said physical connection comprises a communication element selected from the group consisting of Serial Peripheral Interface (“SPI”), MICROWIRE, I²C, +5 V serial, 1-Wire® interface, General Purpose Interface Bus (“GPIB”), RS-232, Peripheral Component Interconnect (“PCI”), memory mapped I/O and Universal Serial Bus (“USB”).

18. The device according to claim 14, wherein said external apparatus comprises at least one of an array assay apparatus, a wash apparatus, an incubator, an array scanner and a personal computer.

19. The device according to claim 12, where said data is encrypted.

20. The device according to claim 12, wherein said data is password protected.

21. The device according to claim 12, wherein said data storage element is capable of storing from about 100 bytes to about 500 megabytes or more of data.

22. The device according to claim 12, wherein said data is at least one of dynamic data and static data.

23. The device according to claim 12, further comprising a sensor for measuring or monitoring a condition, event or process to which said array is subjected.

24. The device according to claim 12, wherein said data storage element is capable of automatically storing data.

25. A system for storing data relating to at least one array, said system comprising:
(a) a substrate having at least one array; and
(b) a device according to claim 1 or claim 12.

26. A method of storing data related to at least one array, said method comprising:
(a) providing a device comprising:
(i) an array holder for holding a substrate having at least one array, and
(ii) a data storage element operatively coupled to said array holder and configured to receive and store data related to said at least one array;
(b) placing a substrate having at least one array in said array holder;
(c) receiving and storing data related to said at least one array in said data storage element;
(d) physically connecting said array holder with at least one external apparatus, wherein data is communicated between said data storage element and said at least one external apparatus via said physical connection.

27. The method according to claim 26, further comprising the steps of measuring at least one condition, event or process related to said at least one array.

28. The method according to claim 26, further comprising the steps of automatically activating or deactivating at least one external apparatus.

29. The method according to claim 26, wherein about 100 bytes to about 500 megabytes or more of said data is stored.

30. The method according to claim 26, wherein said data is at least one of static and dynamic data.

31. The method according to claim 26, wherein said data is stored automatically.

32. The method according to claim 26, wherein said data is communicated automatically.

33. The method according to claim 26, comprising, following exposure of said at least one array to a sample:
reading said at least one array.

34. The method according to claim 33, further comprising forwarding data representing a result of said reading.

35. The method according to claim 34, further comprising the step of transmitting said data to a remote location.

36. The method according to claim 33, further comprising receiving data representing a result of said reading obtained by the method of claim 33.

37. The method according to claim 26, wherein said data is related to at least one of calibration data and the number of times the at least one array is scanned.

38. The method according to claim 26, wherein said data is received and stored substantially concurrently with the occurrence of an array condition, event or process.

39. A method of storing data related to at least one array, said method comprising:
(a) providing a device comprising:
(i) an array holder for holding a substrate having at least one array, and
(ii) a data storage element operatively coupled to said array holder configured to receive and store data related to said at least one array;
(b) placing a substrate having at least one array in said array holder; and
(c) receiving and storing at least one of calibration data and the number of times the array has been scanned in said data storage element.

40. The method according to claim 39, further comprising communicating data between said data storage element and said at least one external apparatus.

41. The method according to claim 40, wherein said data is communicated using at least one of a physical connection and a wireless system.

42. The method according to claim 40, wherein said communication is accomplished automatically.

43. The method according to claim 39, further comprising the steps of automatically activating or deactivating at least one external apparatus.

44. The method according to claim 39, further comprising the steps of measuring at least one condition, event or process related to said at least one array.

45. The method according to claim 39, wherein about 100 bytes to about 500 megabytes or more of said data is stored.

46. The method according to claim 39, further comprising receiving and storing at least one of static and dynamic data.

47. The method according to claim 39, wherein said data is received and stored automatically.

48. The method according to claim 39, comprising, following exposure of said at least one array to a sample:
reading said at least one array.

49. The method according to claim 48, further comprising forwarding data representing a result of said reading.

50. The method according to claim 49, further comprising the step of transmitting said data to a remote location.

51. The method according to claim 48, further comprising receiving data representing a result of said reading obtained by the method of claim 48.

52. The method according to claim 39, wherein said data is received and stored substantially concurrently with the occurrence of an array condition, event or process.

53. A method for processing an array, said method comprising:
(a) providing a device comprising:
(i) an array holder for holding a substrate having at least one array, and
(ii) a data storage element operatively coupled to said array holder configured to receive and store data related to said at least one array;
(b) placing a substrate having at least one array in said array holder;
(c) exposing said at least one array to at least one array process;
(d) receiving and storing data related to said at least one array process in said data storage element; and
(e) physically connecting said array holder with at least one external apparatus, wherein data is communicated between said data storage element and said at least one external apparatus via said physical connection.

54. A method for processing an array, said method comprising:
(a) providing a device comprising:
(i) an array holder for holding a substrate having at least one array, and
(ii) a data storage element operatively coupled to said array holder configured to receive and store data related to said at least one array;
(b) placing a substrate having at least one array in said array holder;
(c) exposing said at least one array to at least one array process; and
(d) receiving and storing at least one of calibration data and the number of times the array has been scanned in said data storage element.

55. A kit for storing data related to at least one array, said kit comprising:
(a) at least one device according to claim 1 and claim 12; and
(b) instructions for using said at least one device.

56. The kit according to claim 55, further comprising at least one of a microprocessor, signal converter and a sensor.

57. The kit according to claim 55, further comprising at least one substrate having at least one array.

58. The kit according to claim 55, further comprising reagents for generating a labeled sample.

59. The kit according to claim 55, wherein said kit further comprises a buffer.

60. The kit according to claim 55, wherein said kit further comprises a wash medium.
